(19) United States
(12) Plant Patent Application Publication
(10) Pub. No.: US 2016/0014949 P1
(43) Pub. Date: Jan. 14, 2016

(54) Dianthus plant named 'KLEDG14178'

(50) ;

(71) Applicant: Klemm+Sohn GmbH & Co. KG, Stuttgart (DE)

(72) Inventors: Nils Klemm, Stuttgart (DE); Martin Glawe, Stuttgart (DE)

(73) Assignee: Klemm+Sohn GmbH & Co. KG, Stuttgart (DE)

(21) Appl. No.: 14/545,531

(22) Filed: May 18, 2015

Related U.S. Application Data
(60) Provisional application No. 61/998,888, filed on Jul. 10, 2014.

(51) Int. Cl. A01H 5/00

(52) U.S. Cl. PLT/284

(57) ABSTRACT

A new dianthus plant named ‘KLEDG14178’ particularly distinguished by purple-red flowers with a dark red center and small, dark blue-green foliage, is disclosed.

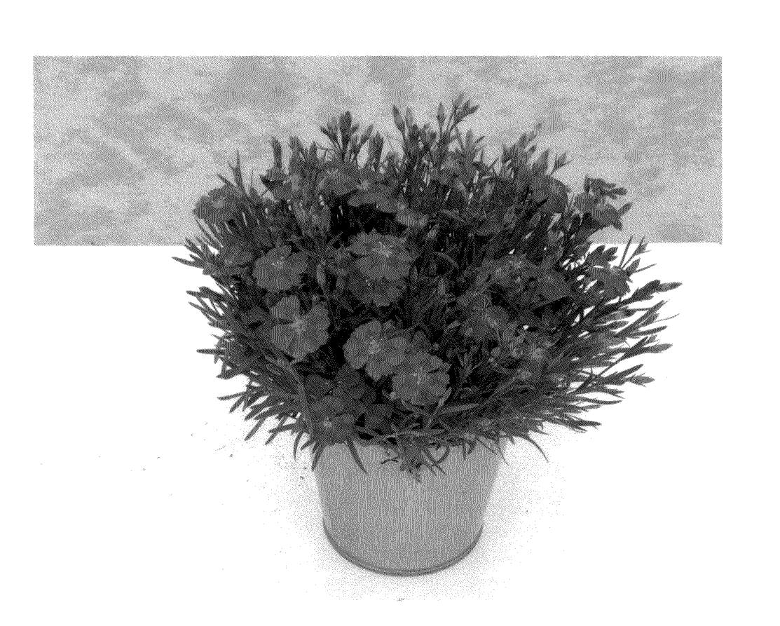

### CROSS REFERENCE TO RELATED APPLICATION

This application claims the benefit of U.S. Provisional Application No. 61/998,888 filed Jul. 10, 2014, the entire contents of which are incorporated herein by reference for all purposes.

### GENUS AND SPECIES

Dianthus sp.

### VARIETY DENOMINATION

‘KLEDG14178’

### BACKGROUND

The present invention comprises a new and distinct variety of Dianthus, botanically known as Dianthus sp. and hereinafter referred to by the variety name ‘KLEDG14178’. ‘KLEDG14178’ originated from a branch mutation from the proprietary dianthus line ‘DSU 2011 0033’ (unpatented).

In June 2011 ‘KLEDG14178’ was discovered as a mutation from the proprietary dianthus line ‘DSU 2011 0033’ in Stuttgart, Germany. ‘KLEDG14178’ was first vegetatively propagated in June 2011 in Stuttgart, Germany by vegetative cuttings. ‘KLEDG14178’ was found to reproduce true to type in successive generations of asexual propagation via vegetative cuttings in Stuttgart, Germany.

### SUMMARY

The following are the most outstanding and distinguishing characteristics of this new variety when grown under normal horticultural practices in Stuttgart, Germany.

1. Purple-red flowers with a dark red center; and

2. Small, dark blue-green foliage.

### DESCRIPTION OF THE PHOTOGRAPH

These new Dianthus plant is illustrated by the accompanying photograph, which shows the overall plant habit of the variety ‘KLEDG14178’ grown in a pot. The photograph is of a 10 to 12 week old plant in Stuttgart, Germany grown under greenhouse conditions in May 2013. The colors shown are as true as can be reasonably obtained by conventional photographic procedures.

### DESCRIPTION OF THE NEW VARIETY

The following detailed descriptions set forth the distinctive characteristics of ‘KLEDG14178’. The data which define these characteristics were collected from asexual reproductions carried out in Stuttgart, Germany. Data was collected on four month old plants grown in a glass greenhouse in Stuttgart, Germany in March, 2014. Color references are to The R.H.S. Colour Chart of The Royal Horticultural Society of London (R.H.S.), 5^{th} edition.
Classification:
   Family.—Caryophyllaceae.
   Species.—Dianthus sp.
   Common name.—Garden carnation.
Parentage: Mutation from the proprietary dianthus ‘DSU 2011 0033’.
Propagation:
   Type.—Vegetative cuttings.
   Time to produce a rooted young plant.—About 20 days.
   Root description.—Small, white, well-branched.
   Rooting habit.—Lanceolate.
Plant description:
   Plant form.—Potted and garden plant, bushy.
   Plant height.—10.0 cm.
   Plant diameter or spread.—18.0 cm.
Lateral branches:
   Length.—6.0 cm.
   Diameter.—1.5 cm.
   Internode length.—0.6 cm.
   Node length.—0.3 cm.
   Node diameter.—0.3 cm.
   Texture.—Smooth.
   Color.—RHS 137B.
Foliage description:
   Arrangement.—Opposite.
   Length.—5.0 cm.
   Width.—0.2 cm.
   Shape.—Lanceolate.
   Apex.—Acute.
   Base.—Obtuse.
   Margin.—Entire.
   Texture.—Upper and lower surfaces: Smooth.
   Color.—Immature and mature leaves: Upper surface: RHS 137B Lower surface: RHS 137C.
   Venation pattern.—Pinnate.
   Venation.—RHS 137D.
Flower description:
   Flower type and habit.—Single, round.
   Fragrance.—Absent.
   Natural flowering season.—Spring to fall.
   Flower longevity.—6 to 8 days.
   Flower buds.—Length: 1.8 cm Diameter: 0.5 cm Shape: Cylindrical Color: RHS 137C.
   Corolla.—Quantity of petals/arrangement: 5, whorled Corolla height: 0.5 cm Petal length: 3.0 cm Petal width: 1.4 cm Petal shape: V-shaped Petal apex: Obtuse. Petal base: Cuspidate Petal margin: Serrate Petal texture: Upper and lower surfaces: Smooth Petal color: Immature: Upper surface: RHS 61B and RHS 59A Lower surface: RHS 61C Mature: Upper surface: RHS 61B and RHS 59B Lower surface: RHS 60C Calyx: Quantity of sepals/arrangement: 5, single Calyx height: 2.0 cm Calyx diameter, at the apex: 0.5 cm Sepal texture: Upper and lower surfaces: Smooth Sepal color: Upper surface: RHS 137C Lower surface: RHS 137D.
Peduncles:
   Length.—6.0 cm.
   Diameter.—1.0 cm.
   Strength.—Moderate.
   Texture.—Smooth.
   Color.—RHS 136B.
Reproductive organs:
   Stamens.—Quantity: Greater than 5 Filament length: 1.0 cm Filament color: RHS 75B. Anther length: 0.15 cm Anther diameter: 0.08 cm Anther color: RHS 91D Pollen amount: Sparse.
   Pistils.—Quantity: 1 Pistil length: 0.8 cm Style length: 0.1 cm Style color: RHS 76D Stigma shape: Filamentous Stigma color: RHS 72A Ovary size: 0.4 cm Ovary shape: Ovate Ovary color: RHS 139D Fruits/seeds: None observed.
Disease and insect/pest resistance: No special observations were made

### COMPARISON WITH PARENTAL LINE AND KNOWN VARIETY

When ‘KLEDG14178’ is compared to the parental line, the following differences are noted in Table 1:

TABLE 1
Comparison with Parental Line

| | Variety | Female parent |
|---|---|---|
| Trait | ′KLEDG14178′ | ′DSU 2011 0033′ |
| Flower color | Purple-Red with a | Dark purple with |
| | dark red center | dark center |

When ‘KLEDG14178’ is compared to the commercial line ‘Kahori’ (patent status unknown), the following differences are noted in Table 2:

TABLE 2
Comparison with Similar Variety

| | Variety | Commercial line |
|---|---|---|
| Trait | ′KLEDG14178′ | ′Kahori′ |
| Flower color | Purple-red with a dark red center | Purple and blue |
| Color of foliage | Dark green-blue | Green |
| Size of leaves | Small | Medium |

We claim:

1. A new and distinct variety of Dianthus plant named ‘KLEDG14178’ as illustrated and described herein.
